(19) United States
(12) Patent Application Publication
(10) Pub. No.: US 2010/0173890 A1
(43) Pub. Date: Jul. 8, 2010

(54) Compounds, Process for their Preparation, Intermediates, Pharmaceutical Compositions and their use in the Treatment of 5-HT6 Mediated Disorders such as Alzheimer's Disease, Cognitive Disorders, Cognitive Impairment Associated with Schizophrenia, Obesity and Parkinson's Disease

(75) Inventors: Gunnar Nordvall, Sodertalje (SE); Fernando Sehgelmeble, Sodertalje (SE)

Correspondence Address: ASTRA ZENECA PHARMACEUTICALS LP;GLOBAL INTELLECTUAL PROPERTY, 1800 CONCORD PIKE, WILMINGTON, DE, 19850-5437, US

(73) Assignee: AstraZeneca AB, Sodertalje (SE)

(21) Appl. No.: 11/993,378

(22) PCT Filed: Jul. 3, 2006
(86) PCT No.: PCT/SE06/00828

§ 371 Date: Dec. 20, 2007

(30) Foreign Application Priority Data
Jul. 5, 2005 (SE) 0501580-5

(51) Int. Cl. A61K 31/553; C07D 267/14; C07D 498/04; C07D 223/16; A61K 31/5513; A61P 25/28

(52) U.S. Cl. 514/211.1; 540/552; 540/548; 540/593; 514/213.01; 514/211.09

(57) ABSTRACT

The present invention relates to new compounds of formula I, or salts, solvates or solvated salts thereof, process for their preparation and to new intermediates used in the preparation thereof, pharmaceutical compositions containing said compounds and to the use of said compounds in the treatment of 5-HT6 mediated disorders such as Alzheimer's disease, cognitive disorders, cognitive impairment associated with schizophrenia, obesity and Parkinson's disease.

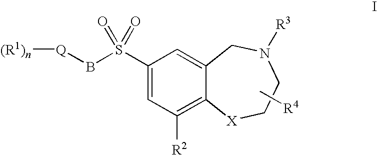

### FIELD OF THE INVENTION

The present invention relates to new compounds, to pharmaceutical compositions containing said compounds and to the use of said compounds in therapy. The present invention further relates to processes for the preparation of said compounds and to the use of intermediates in the preparation thereof.

### BACKGROUND OF THE INVENTION

Serotonin (5-hydroxy-tryptamine) (5-HT) receptors play an important role in many is physiological and pathological functions like anxiety, sleep regulation, aggression, feeding and depression. The 5-HT receptors are distributed throughout the body and can be divided into seven different 5-HT receptor subtypes, i.e. 5-HT1-5-HT7, with different properties. The 5-HT6 receptor is mostly found in the central nervous system (CNS). From in situ hybridization studies it is known that the 5-HT6 receptor in rat brain is localized in areas like striatum, nucleus accumbens, olfactory tubercle and hippocampal formation (Ward et al., Neuroscience, 64, p 1105-1111, 1995).

Scientific research has revealed a potential therapeutic use for modulators of the 5-HT6 receptor, especially with regard to various CNS disorders. Blocking 5-HT6 receptor function has been shown to enhance cholinergic transmission (Bentley et al, Br J Pharmacol 126: 1537-1542, 1999; Riemer et al J Med Chem 46, 1273-1276). 5-HT6 antagonist have also been shown to reverse cognitive deficits in in vivo cognition models induced by the muscarinic antagonist scopolamine (Woolley et al. Phychopharmacolgy, 170, 358-367, 2003; Foley et al. Neuropsychopharmacology, 29 93-100, 2004)

Studies have shown that 5-HT6 antagonists increase levels of glutamate and aspartate in the frontal cortex and dorsal hippocampus as well as acetylcholine in the frontal cortex. These neurochemicals are known to be involved in memory and cognition (Dawson et al., Neuropsychopharmacology., 25(5), p 662-668, 2001) (Gerard et al., Brain Res., 746, p 207-219, 1997) (Riemer et al J Med Chem 46(7), p 1273-1276, 2003).

Acetylcholinesterase inhibitors increase the levels of acetylcholine in the CNS and are used in the treatment of cognitive disorders such as Alzheimer's disease. 5-HT6 antagonists may therefore be used in the treatment of cognitive disorders.

Studies have also shown that 5-HT6 antagonist increases the level of dopamine and noradrenaline in the medial prefrontal cortex (Lacroix et al. Synapse 51, 158-164, 2004). In addition, 5-HT6 receptor antagonists have been shown to improve performance in the attentional set shifting task (Hatcher et al. Psychopharmacology 181(2):253-9, 2005).

Therefore, 5-HT6 ligands are expected to be useful in the treatment of disorders where cognitive deficits are a feature, such as schizophrenia. Several antidepressants and atypical antipsychotics bind to the 5-HT6 receptor and this may be a factor in their profile of activities (Roth et al., J. Pharm. Exp. Therapeut., 268, 1402-1420, 1994; Sleight et al., Exp. Opin. Ther. Patents, 8, 1217-1224, 1998; Kohen et al., J. Neurochem., 66(1), p 47-56, is 1996; Sleight et al. Brit. J. Pharmacol., 124, p 556-562, 1998; Bourson et al., Brit. J. Pharmacol., 125, p 1562-1566, 1998).

Stean et al., (Brit. J. Pharmacol. 127 Proc. Supplement 131P, 1999) have described the potential use of 5-HT6 modulators in the treatment of epilepsy. 5-HT6 receptors have also been linked to generalized stress and anxiety states (Yoshioka et al., Life Sciences, 62, 17/18, p 1473-1477, 1998). 5-HT6 agonists have been shown to elevate levels of GABA in brain regions associated with anxiety and shown positive effects in models predictive of obsessive-compulsive disorder (Schechter et al. NeuroRx. 2005 October; 2(4): 590-611). The use of modulators for this receptor is therefore expected for a wide range of CNS disorders.

Pullagurla et al (Pharmacol Biochem Behay. 78(2):263-8, 2004) have described the potential use of 5-HT6 antagonists in disorders were the dopamine transmission is affected, for example a combination between a 5-HT6 antagonist and a dopamine enhancer for example levodopa/carbidopa or amantidine would be expected to have advantages compared to administration of only a dopamine enhancer.

Moreover, a reduction in food intake in rats has been reported using 5-HT6 receptor modulators (Bentley et al., Br. J. Pharmacol. Suppl. 126, P66, 1999; Bentley et al. J. Psychopharmacol. Supl. A64, 255, 1997; Pendharkar et al Society for Neuroscience, 2005). 5-HT6 receptor modulators may therefore also be useful in the treatment of feeding disorders like anorexia, obesity, bulimia and similar disorders and also type 2 diabetes.

### DETAILED DESCRIPTION OF THE INVENTION

The object of the present invention is to provide compounds exhibiting a modulating activity at the 5-hydroxy-tryptamine 6 receptor.

The present invention provides compounds of formula I

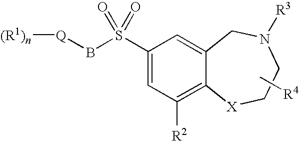

wherein:

Q is C₆₋₁₀arylC₀₋₆alkyl, C₅₋₁₁heteroarylC₀₋₆alkyl, C₃₋₇cycloalkylC₀₋₆alkyl, C₃₋₇heterocycloalkyl or C₁₋₃alkyl;

R¹ is hydrogen, hydroxyl, halogen, C₁₋₆alkyl, C₂₋₆alkenyl, C₂₋₆alkynyl, C₁₋₆alkoxy, N(R⁸)₂, C₆₋₁₀arylC₀₋₃alkyl, C₅₋₆heteroarylC₀₋₃alkyl, C₁₋₆haloalkyl, C₁₋₆haloalkylO, R⁶OC₀₋₆alkyl, CN, SR⁶, R⁶SO₂C₀₋₃allcyl, SOR⁶, R⁶CON(R⁷)C₀₋₃alkyl, NR⁷SO₂R⁶, COR⁶, COOR⁶, OSO₂R⁶, (R⁷)₂NCOC₀₋₃alkyl, SO₂N(R⁷)₂, N(R⁷)CON(R⁷)₂, NO₂ or oxo;

n is 0, 1, 2, 3 or 4;

B is O, N(R⁵), or B is N in a C₅₋₁₁heteroaryl;

X is O, CH₂ or NR¹⁰;

R² is hydrogen, hydroxyl, halogen, C₁₋₆alkyl, C₁₋₆alkoxy, C₁₆haloalkyl, C₁₋₆haloallcylO, R⁹OC₀₋₆alkyl, CN, SR⁸, SO₂R⁹, SOR⁹, N(R⁸)COR⁹, N(R⁸)SO₂R⁹, COOR⁹, OSO₂R⁹, CON(R⁸)₂ or SO₂N(R⁹)₂;

R³ is hydrogen, C₁₋₁₀alkyl, C₁₋₆haloalkyl or R⁹OC₁₋₆allcyl;

R⁴ is hydrogen, C₁₋₅allcyl, C₁₋₅haloalkyl, C₁₋₅alkoxy or C₁₋₅haloalkoxy and may be substituted by one or more groups selected independently from halogen, hydroxyl, cyano, C₁₋₃alkyl and C₁₋₃acyloxy, or

R³ and R⁴ form together a C₃₋₇heterocycloalkyl, which may be substituted by one or more groups selected independently from hydrogen, halogen, C₁₋₆alkyl, C₁₋₆haloalkyl, COR⁹, SO₂R⁹, OR⁹, cyano, oxo and SO₂N(R⁸)₂;

R⁵ is hydrogen, C₁₋₆alkyl, R⁹OC₁₋₆allcyl, C₁₋₆haloalkyl or C₁₋₆cyanoalkyl;

R⁶ is C₁₋₆alkyl, C₆₋₁₀arylC₀₋₃alkyl, C₅₋₆heteroarylC₀₋₃alkyl, C₃₋₇cycloalkylC₀₋₃alkyl or C₁₋₃haloalkyl;

R⁷ is hydrogen, C₁₋₆alkyl, C₁₋₆haloalkyl, C₃₋₇cycloallcylC₀₋₃alkyl, C₆₋₁₀arylC₀₋₃alykl, or C₅₋₆heteroarylC₀₋₃alkyl, or

R⁶ and R⁷ form together a C₅₋₆heteroaryl or C₃₋₇heterocycloalkyl,

whereby any aryl and heteroaryl under R¹, R⁶ and R⁷ may be substituted by one or more groups selected independently from hydrogen, halogen, hydroxyl, C₁₋₆haloalkyl, CN, OR⁸, C₁₋₆allcyl, oxo, SR⁸, CON(R⁸)₂, N(R⁸)COR⁹, SO₂R⁹, SOR⁹, N(R⁸)₂ and COR⁹;

R⁸ is hydrogen, C₁₋₆alkyl, C₁₋₆cyanoalkyl or C₁₋₆haloalkyl; and

R⁹ is C₁₋₆allcyl, C₁₋₆cyanoalkyl or C₁₋₆haloalkyl;

R⁸and R⁹ form together a C₃₋₇heterocycloalkyl which may be substituted by one or more groups selected independently from hydrogen, halogen, hydroxyl, C₁₋₃allcyl, C₁₋₃alkoxy and cyano; and

R¹⁰ is H, C₁₋₆alkyl, C₁₋₆haloalkyl, COR¹¹ or SO₂R¹¹;

or salts, solvates or solvated salts thereof.

In another aspect of the invention there is provided compounds of formula I, wherein:

Q is C₆₋₁₀arylC₀₋₆alkyl or C₅₋₁₁heteroarylC₀₋₆alkyl;

R¹ is hydrogen, halogen, C₁₋₆alkyl, C₆₋₁₀arylC₀₋₃alkyl, C₅₋₆heteroarylC₀₋₃alkyl, C₁₋₆haloalkyl, CN or R⁶OC₀₋₆alkyl;

n is 1 or 2;

B is O or N(R⁵);

X is O, CH₂ or NR¹⁰;

R² is hydrogen, hydroxyl, halogen, C₁₋₆alkyl, C₁₋₆alkoxy, C₁₋₆haloalkyl, C₁₋₆haloalkylO;

R³ is hydrogen, C₁₋₁₀alkyl, C₁₋₆haloalkyl or R⁹OC₁₋₆alkyl;

R⁴ is hydrogen, C₁₋₅alkyl, C₁₋₅haloalkyl, C₁₋₅alkoxy or C₁₋₅haloalkoxy and may be substituted by one or more groups selected independently from halogen, hydroxyl, cyano, C₁₋₃alkyl and C₁₋₃alkoxy, or

R³ and R⁴ form together a C₃₋₇heterocycloalkyl, which may be substituted by one or more groups selected independently from hydrogen, halogen, C₁₋₆allcyl, C₁₋₆haloalkyl, COR⁹, SO₂R⁹, OR⁹, cyano, oxo and SO₂N(R⁸)₂; and

R⁵ is hydrogen, C₁₋₆alkyl, C₁₋₆haloalkyl or C₁₋₆cyanoalkyl;

or salts, solvates or solvated salts thereof.

In yet another aspect of the invention there is provided compounds of formula I, wherein:

Q is C₆₋₁₀arylC₀₋₆alkyl;

R¹ is halogen;

n is 1 or 2;

B is O or N(R⁵);

X is O, CH₂ or NR¹⁰;

R² is hydrogen or halogen;

R³ is hydrogen or C₁₋₁₀alkyl;

R⁴ is hydrogen or C₁₋₅alkyl, or

R³ and R⁴ form together a C₃₋₇heterocycloalkyl; and

R⁵ is hydrogen;

or salts, solvates or solvated salts thereof.

In yet another aspect of the invention there is provided compounds of formula I wherein Q is phenyl or naftyl, substituted by R¹ whereby R¹ is hydrogen, halogen, C₁₋₆alkyl, C₆₋₁₀arylC₀₋₃alkyl, C₅₋₆heteroarylC₀₋₃alkyl, C₁₋₆haloalkyl, CN or R⁶OC₀₋₆alkyl.

In another embodiment R¹ is a halogen such as chloro, bromo, iodo and fluoro.

In yet a further embodiment R¹ is methyl, ethyl, propyl, butyl or pentyl.

A further embodiment of the invention relates to compounds selected from the group consisting of:

N-(3-Bromophenyl)-9-chloro-4-isopropyl-2,3,4,5-tetrahydro-1,4-benzoxazepine-7-sulfonamide;

9-Chloro-N-(3-chlorophenyl)-2,3,11,11a-tetrahydro-1H,5H-pyrrolo[2,1-c][1,4]benzoxazepine-7-sulfonamide;

N-(3-Chlorophenyl)-2,3,4,5-tetrahydro-1,4-benzoxazepine-7-sulfonamide;

N-(2,3-Dichlorophenyl)-2,3,4,5-tetrahydro-1,4-benzoxazepine-7-sulfonamide;

N-(4-Chloro-1-naphthyl)-2,3,4,5-tetrahydro-1,4-benzoxazepine-7-sulfonamide; and

2-Bromophenyl 2,3,4,5-tetrahydro-1H-2-benzazepine-8-sulfonate,

or salts, solvates or solvated salts thereof.

Listed below are definitions of various terms used in the specification and claims to describe the present invention.

For the avoidance of doubt it is to be understood that where in this specification a group is qualified by ‘hereinbefore defined’, ‘defined hereinbefore’ or ‘defined above’ the said group encompasses the first occurring and broadest definition as well as each and all of the other definitions for that group.

For the avoidance of doubt it is to be understood that in this specification ‘C₁₋₆’ means a carbon group having 1, 2, 3, 4, 5 or 6 carbon atoms.

In this specification, unless stated otherwise, the term “alkyl” includes both straight and branched chain alkyl groups and may be, but are not limited to methyl, ethyl, n-propyl, i-propyl, n-butyl, i-butyl, s-butyl, t-butyl, n-pentyl, i-pentyl, neo-pentyl, n-hexyl, i-hexyl, etc. The term C₁₋₁₀alkyl having 1 to 10 carbon atoms and may be but are not limited to methyl, ethyl, n-propyl, i-propyl, n-butyl, i-butyl, s-butyl, tert-butyl, n-pentyl, i-pentyl, neo-pentyl, etc.

The term ‘C₀’ means a bond or does not exist. For example when “arylC₀alkyl” is equivalent with “aryl”, “C₂alkylOC₀alkyl” is equivalent with “C₂alkylO”.

In this specification, unless stated otherwise, the term “alkenyl” includes both straight and branched chain alkenyl groups. The term “C₂₋₆alkenyl” having 2 to 6 carbon atoms and one or two double bonds, may be, but is not limited to vinyl, allyl, propenyl, butenyl, crotyl, pentenyl, hexenyl, and a butenyl group may for example be buten-2-yl, buten-3-yl or buten-4-yl.

In this specification, unless stated otherwise, the term “alkynyl” includes both straight and branched chain alkynyl groups. The term “C₂₋₆alkynyl” having 2 to 6 carbon atoms and one or two trippel bonds, may be, but is not limited to etynyl, propargyl, pentynyl or hexynyl and a butynyl group may for example be butyn-3-yl or butyn-4-yl.

In this specification, unless stated otherwise, the term “cycloalkyl” refers to an optionally substituted, partially or completely saturated cyclic hydrocarbon ring system. The term “C₃₋₇cycloalkyl” may be, but is not limited to cyclopropyl, cyclobutyl, cyclopentyl, cyclohexyl, cycloheptyl or cyclopentenyl.

The term “alkoxy”, unless stated otherwise, refers to radicals of the general formula —O—R, wherein R is selected from a hydrocarbon radical. The term “C₁₋₆alkoxy” may include, but is not limited to methoxy, ethoxy, propoxy, isopropoxy, butoxy, t-butoxy, isobutoxy, cyclopropylmethoxy, allyloxy, propargyloxy, pentoxy, isopentoxy, etc.

In this specification, unless stated otherwise, the term “amine” or “amino” refers to radicals of the general formula —NRR′, wherein R and R′ are selected independently from hydrogen or a hydrocarbon radical. The term ‘N(R⁵)’ refers to a group wherein R⁵ may the same or different.

The term “heterocycloalkyl” denotes a non-aromatic, partially or completely saturated hydrocarbon group, which contains one ring and at least one heteroatom. Examples of said heterocycle include, but are not limited to pyrrolidinyl, pyrrolidonyl, piperidinyl, piperazinyl, morpholinyl, oxazolyl, 2-oxazolidonyl or tetrahydrofuranyl.

In this specification, unless stated otherwise, the term “aryl” refers to an optionally substituted monocyclic or bicyclic hydrocarbon ring system with at least one unsaturated aromatic ring. Examples of “aryl” may be, but are not limited to phenyl, naphthyl or tetralinyl.

In this specification, unless stated otherwise, the term “heteroaryl” refers to an optionally substituted monocyclic or bicyclic hydrocarbon ring system with at least one unsaturated ring and containing at least one heteroatom selected independently from N, O or S.

Examples of “heteroaryl” may be, but are not limited to pyridyl, pyrrolyl, furyl, thienyl, imidazolyl, oxazolyl, isoxazolyl, thiazolyl, pyrazolyl, benzofuryl, indolyl, isoindolyl, benzimidazolyl, pyridazinyl, pyrimidinyl, pyrazinyl, tetrazolyl, triazolyl, quinazolinyl or isotiazolyl. For the avoidance of doubt, a C₅heteroaryl refers to a 5 membered aromatic ring system containing at least one heteroatom.

In this specification, unless stated otherwise, the terms “arylalkyl” and “heteroarylalkyl” refer to a substituent that is attached via the alkyl group to an aryl or heteroaryl group.

In this specification, unless stated otherwise, the terms “halo” and “halogen” may be fluoro, iodo, chloro or bromo.

In this specification, unless stated otherwise, the term “haloalkyl” means an alkyl group as defined above, which is substituted with halo as defined above. The term “C₁₋₆haloalkyl” may include, but is not limited to fluoromethyl, difluoromethyl, trifluoromethyl, fluoroethyl, difluoroethyl or bromopropyl. The term “C₁₋₆haloalkylO” may include, but is not limited to fluoromethoxy, difluoromethoxy, trifluoromethoxy, fluoroethoxy or difluoroethoxy.

The present invention relates to the compounds of formula I as hereinbefore defined as well as to the salts, solvates or solvated salts thereof. Salts for use in pharmaceutical compositions will be pharmaceutically acceptable salts, but other salts may be useful in the production of the compounds of formula I.

A suitable pharmaceutically acceptable salt of the compounds of the invention is, for example, an acid-addition salt, for example a salt with an inorganic or organic acid. In addition, a suitable pharmaceutically acceptable salt of the compounds of the invention is an alkali metal salt, an alkaline earth metal salt or a salt with an organic base. Other pharmaceutically acceptable salts and methods of preparing these salts may be found in, for example, Remington's Pharmaceutical Sciences (18^{th} Edition, Mack Publishing Co.).

Some compounds of formula I may have chiral centres and/or geometric isomeric centres (E- and Z-isomers), and it is to be understood that the invention encompasses all such optical, diastereoisomeric and geometric isomers.

The invention also relates to any and all tautomeric forms of the compounds of formula I.

Methods of Preparation

General Procedure

Throughout the following description of such processes it is to be understood that, where appropriate, suitable protecting groups will be added to, and subsequently removed from, the various reactants and intermediates in a manner that will be readily understood by one skilled in the art of organic synthesis. Conventional procedures for using such protecting groups as well as examples of suitable protecting groups are described, for example, in “Protective Groups in Organic Synthesis”, T. W. Green, P. G. M. Wuts, Wiley-Interscience, New York, (1999). It is also to be understood that a transformation of a group or substituent into another group or substituent by chemical manipulation can be conducted on any intermediate or final product on the synthetic path toward the final product, in which the possible type of transformation is limited only by inherent incompatibility of other functionalities carried by the molecule at that stage to the conditions or reagents employed in the transformation. Such inherent incompatibilities, and ways to circumvent them by carrying out appropriate transformations and synthetic steps in a suitable order, will be readily understood to the one skilled in the art of organic synthesis. Examples of transformations are given below, and it is to be understood that the described transformations are not limited only to the generic groups or substituents for which the transformations are exemplified. References and descriptions on other suitable transformations are given in “Comprehensive Organic Transformations—A Guide to Functional Group Preparations” R. C. Larock, VHC Publishers, Inc. (1989). References and descriptions of other suitable reactions are described in textbooks of organic chemistry, for example, “Advanced Organic Chemistry”, March, 4^{th} ed. McGraw Hill (1992) or, “Organic Synthesis”, Smith, McGraw Hill, (1994). Techniques for purification of intermediates and final products include for example, straight and reversed phase chromatography on column or rotating plate, recrystallisation, distillation and liquid-liquid or solid-liquid extraction, which will be readily understood by the one skilled in the art.

The definitions of substituents and groups are as in formula I except where defined differently. The specific sequence of reactions depicted under “General procedure” is not critical. For many of the compounds described the order of the reaction steps may be varied. The reactions were run until determined complete by LC-UV, LC-MS, TLC or NMR.

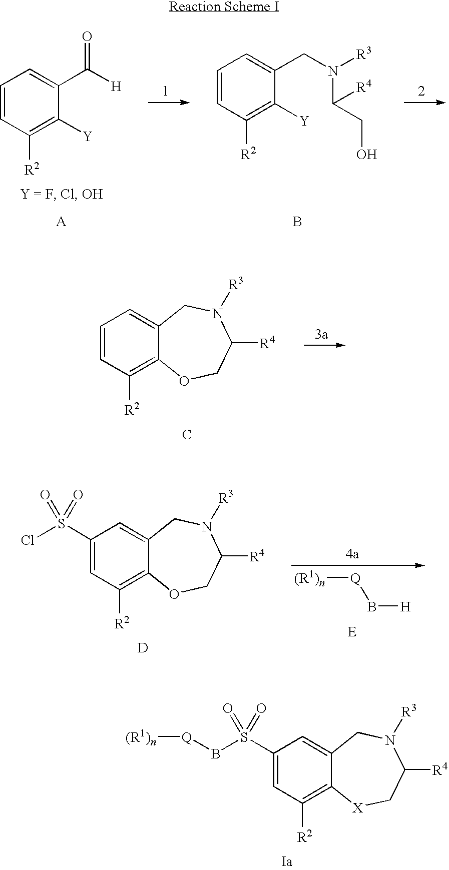

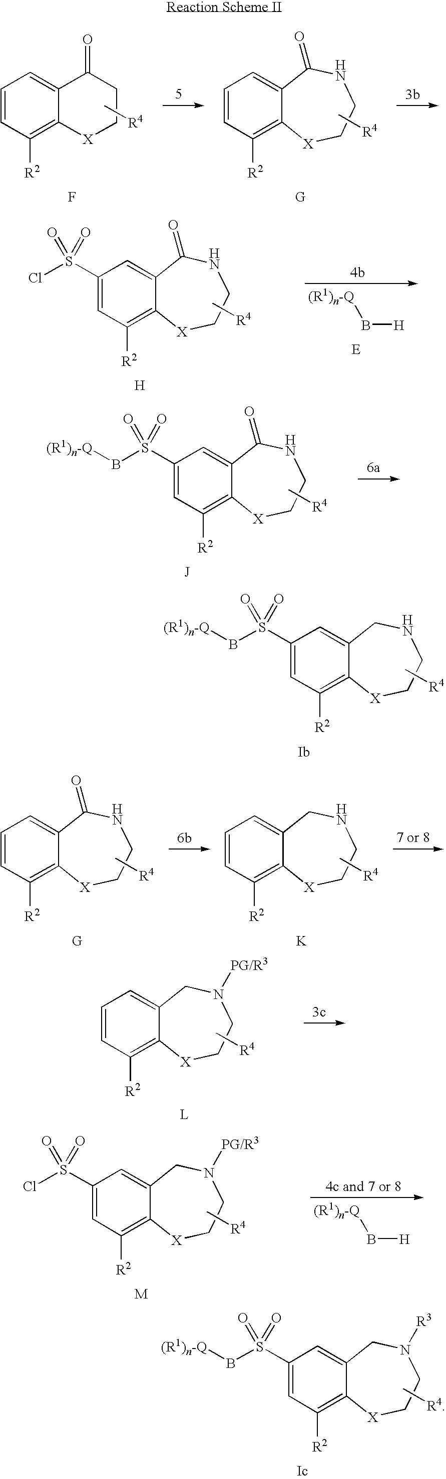

Step 1

A compound B may be prepared from a compound A using reductive amination. Typically A may be mixed with a carbonyl compound such as an aldehyde or a ketone in the presence of a reducing agent such as sodium borohydride, sodium cyanoborohydride, sodium triacetoxyborohydride or hydrogen in the presence of a suitable catalyst such as for example described in “Advanced Organic Chemistry, Reactions, Mechanisms and Structure”, J. March, John Wiley & Sons, New York, 1992. An acid such as formic acid or acetic acid may be added to control the pH of the reaction. The reaction may be performed in a solvent such as water, methanol, ethanol, dichloromethane, THF, formic acid, acetic acid or mixtures thereof at temperatures between 0° C. and the reflux temperature of the solvent, preferably at room temperature. The reaction mixture may be either worked up by extraction and then purified by column chromatography or the reaction mixture may be concentrated and purified by column chromatography.

Step 2

A compound B may be transformed into a compound C via intramolecular aromatic nucleophilic substitution where Y═F or Cl. Typically a compound B is dissolved in a solvent such as THF, dioxane or DMF and a base such as sodium hydride or sodium methoxide is added. The reaction may be performed at temperatures between room temperature and the reflux temperature of the solvent for reaction times between 1 and 24 h. The product may be isolated by extraction, precipitation or column chromatography. Alternatively, when Y═OH, an intramolecular ringclosure of Mitsunobo type may be used.

Typically a compound B may be dissolved in a solvent such as DMF, THF or dichloromethane or mixtures thereof. A phosphine compound such as triphenylphosphine or tributylphosphine and an activating agent such as diethyl azodicarboxylate or diisopropyl azodicarboxylate are added, preferably at temperatures between −10° C. and room temperature. The reaction may be performed at temperatures between −15° C. and the reflux temperature of the solvent, preferably at room temperature for reaction times between 1 and 24 h. The product may be isolated by extraction, precipitation or column chromatography.

Step 3

A compound C may be transformed into a compound D by chlorosulfonylation. Typically compound C may be dissolved in a solvent such as dichloromethane, chloroform or ethyl acetate and cooled to between −72° C. and 0° C. The reaction may also be run neat in chlorosulfonic acid. Chlorosulfonic acid, optionally diluted in a solvent such as chloroform, may be added dropwise while cooling. The reaction may be stirred for 10 min-1 h while cooling and then let to room temperature or heated to the reflux temperature of the solvent for 1-100 h, typically for 1-5 h. Optionally a chlorinating agent such as thionyl chloride may be added to the reaction mixture. The reaction may be quenched by adding the reaction mixture to ice water, optionally containing a base such as sodium bicarbonate and the raw product may be isolated by extraction and used without further purification or if stable enough, purified by column chromatography.

To ensure complete transformation of a compound C into a compound D via the sulfonic acid, the crude may be dissolved in a solvent such as chloroform or toluene and a chlorinating agent such as thionyl chloride or oxalyl chloride may be added. Optionally a catalytic amount of DMF may be added and the mixture may be heated to between 25° C. and the reflux temperature of the solvent. The workup and purification may be performed as in the previous section.

The same reaction conditions may be used for the transformation of a compound G to a compound H or a compound L to a compound M.

Step 4

A compound Ia may be prepared by the reaction of a compound D with a compound of formula E. Typically a compound D may be reacted with a compound E in the presence of an organic base such as pyridine, triethylamine or diisopropylethylamine or an inorganic base such as sodium hydroxide or potassium carbonate in a solvent such as dichloromethane, chloroform, acetonitrile, DMF, THF or mixtures thereof at a temperature between 0° C. and the reflux temperature of the solvent, preferably at room temperature. The product may be isolated by column chromatography or by extraction followed by column chromatography.

The same procedures may be used to transform a compound H into a compound J or a compound M into a compound Ic.

Step 5

A compound F may be transformed into a compound G via the Schmidt rearrangement.

Compound F and sodium azide may be dissolved in a solvent such as benzene, TFA or acetic acid. Sulfuric acid may be added at temperatures below 5° C., typically between −10° C. and 5° C. The reaction may be performed at temperatures between room temperature and the reflux temperature of the solvent. The mixture may then be poured onto ice or water and the mixture may be made basic with a base such as ammonia, potassium carbonate or sodium hydroxide. The mixture may be stirred at room temperature for 1-20 h and the product may be isolated by extraction, precipitation or column chromatography.

Step 6

The reduction of compound J to compound Ib may be performed with a reducing agent such as borane or lithium aluminum hydride in a solvent such as tetrahydrofuran or diethyl ether at temperatures between 0° C. and the reflux temperature of the solvent, preferably between 25° C. and the reflux temperature. The product may be isolated by column chromatography or by extraction.

The same procedures may be used to transform a compound G into a compound K.

Step 7

A compound K may be transformed into a compound L using standard protecting groups. Conventional procedures for using such protecting groups, as well as examples of suitable protecting groups are described in, for example, “Protective Groups in Organic Synthesis” T. W. Green, P. G. M. Wuts, Wiley-Interscience, New York, 1999.

Step 8

A compound Ic2 where R³ is not H may be prepared from a compound Ic1 where R³ is H by alkylation with a compound R³Y² where Y² may be a suitable leaving group such as a halogen, mesylate or Inflate, such as for example described in “Comprehensive Organic Transformations, a Guide to Functional Group Preparation”, R. C. Larock, John Wiley & sons, New York, 1999. Typically, Ic1 and R³Y² are mixed in a solvent such as DMF, ethanol, dichloromethane or toluene in the presence of a base such as sodium bicarbonate, sodium carbonate, potassium carbonate, triethylamine or diisopropylethylamine and optionally, if Y═Cl, Br, a catalytic amount of potassium iodide. The reaction may be performed at temperatures between 25° C. and the reflux temperature of the solvent and the reaction time may be between 1 and 100 hours. The reaction mixture may be either worked up by extraction and then purified by column chromatography or the reaction mixture may be concentrated and purified by column chromatography. The reaction temperature may be elevated above the reflux temperature of the solvent and reaction times shortened by the use of microwave heating. Alternatively, a compound Ic2 may be prepared from a compound Ic1 using reductive amination Typically compound Ic1 may be mixed with a carbonyl compound such as an aldehyde or a ketone in the presence of a reducing agent such as sodium borohydride, sodium cyanoborohydride, sodium triacetoxyborohydride or hydrogen in the presence of a suitable catalyst such as for example described in “Advanced Organic Chemistry, Reactions, Mechanisms and Structure”, J. March, John Wiley & Sons, New York, 1992. An acid such as formic acid or acetic acid may be added to control the pH of the reaction. The reaction may be performed in a solvent such as water, methanol, ethanol, dichloromethane, THF, formic acid, acetic acid or mixtures thereof at temperatures between 0° C. and the reflux temperature of the solvent, preferably at room temperature. The reaction mixture may be either worked up by extraction and then purified by column chromatography or the reaction mixture may be concentrated and purified by column chromatography.

A compound Ic2 may also be prepared from a compound Ic1 by first preparing the amide or carbamate followed by reduction using an appropriate reducing agent. The amide may for example be prepared by reaction of Ic1 with an acid chloride or with a carboxylic acid in the presence of a coupling reagent, such as for example described in “Comprehensive Organic Transformations, a Guide to Functional Group Preparation”, R. C. Larock, John Wiley & sons, New York, 1999. The carbamate may be prepared by the reaction of an alkylchloroformate with a compound Ic1 in a solvent such as dichloromethane in the presence of a base such as triethylamine or pyridine at temperatures between 0° C. and the reflux temperature of the solvent. The reduction of the carbamate or the amide may be performed with a reducing agent such as lithium aluminum hydride in a solvent such as tetrahydrofuran or diethyl ether at temperatures between 0° C. and the reflux temperature of the solvent, preferably between 25° C. and the reflux temperature. The reduction of the amide may also be performed using borane as the reducing agent.

Intermediates

One embodiment relates to intermediates of formula M1

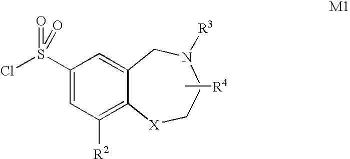

wherein X, R², R³ and R⁴ are defined as above.

Another embodiment relates to the use of the compound of formula M1 in the preparation of compounds of formula I.

Pharmaceutical Composition

According to one embodiment of the present invention there is provided a pharmaceutical composition comprising as active ingredient a therapeutically effective amount of the compound of formula I, or salts, solvates or solvated salts thereof, in association with one or more pharmaceutically acceptable diluents, excipients and/or inert carriers.

The composition may be in a form suitable for oral administration, for example as a tablet, pill, syrup, powder, granule or capsule, for parenteral injection (including intravenous, subcutaneous, intramuscular, intravascular or infusion) as a sterile solution, suspension or emulsion, for topical administration e.g. as an ointment, patch or cream, for rectal administration, e.g. as a suppository, or for inhalation.

In general, the above compositions may be prepared in a conventional manner using one or more conventional excipients, pharmaceutical acceptable diluents and/or inert carriers. Suitable daily doses of the compounds of formula I in the treatment of a mammal, including man, are approximately 0.01 to 250 mg/kg bodyweight at peroral administration and about 0.001 to 250 mg/kg bodyweight at parenteral administration.

The typical daily dose of the active ingredient varies within a wide range and will depend on various factors such as the relevant indication, severity of the illness being treated, the route of administration, the age, weight and sex of the patient and the particular compound being used, and may be determined by a physician.

Medical Use

Interestingly, it has been found that the compounds according to the present invention are useful in therapy. The compounds of formula I, or salts, solvates or solvated salts thereof, as well as their corresponding active metabolites or prodrugs, exhibit a high degree of potency and selectivity for 5-hydroxy-tryptamine 6 (5-HT6) receptors. Accordingly, the compounds of the present invention are expected to be useful in the treatment of conditions associated with excessive activation of 5-HT6 receptors.

The compounds of formula I are expected to be suitable for the treatment of disorders relating to or affected by the 5-HT6 receptor including cognitive, personality, behaviour, psychiatric and neurodegenerative disorders.

Examples of such disorder may be selected from the group comprising of Alzheimer's disease anxiety, depression, convulsive disorders such as epilepsy, personality disorders, obsessive compulsive disorders, migraine, cognitive disorders such as memory dysfunction, sleep disorders, feeding disorders such as anorexia, obesity, bulimia, panic attacks, withdrawal from drug abuse, schizophrenia, cognitive impairment associated with schizophrenia, attention deficit hyperactive disorder (ADHD), attention deficit disorder (ADD), dementia, memory loss, disorders associated with spinal trauma and/or head injury, stroke, diabetes type 2, binge disorders, bipolar disorders, psychoses, Parkinson's disease, Huntington's disease, neurodegenerative disorders characterized by impaired neuronal growth, and pain.

Further relevant disorders may be selected from the group comprising gastro-intestinal disorders such as gastro-esophageal reflux disease (GERD) and irritable bowel syndrome (IBS).

The compounds may also be used for treatment of tolerance to 5-HT6 activators.

One embodiment of the invention relates to the compounds of formula I as hereinbefore defined, for use in therapy.

Another embodiment of the invention relates to the compounds of formula I as hereinbefore defined, for use in treatment of 5-HT6 mediated disorders.

A further embodiment of the invention relates to the compounds of formula I as hereinbefore defined, for use in treatment of Alzheimer's disease.

Another embodiment of the invention relates to the compounds of formula I as hereinbefore defined, for use in treatment of cognitive disorders such as for example cognitive impairment associated with schizophrenia.

Yet a further embodiment of the invention relates to the compounds of formula I as hereinbefore defined, for use in treatment of obesity.

One embodiment of the invention relates to the compounds of formula I as hereinbefore defined, for use in treatment of Parkinson's disease.

Another embodiment of the invention relates to the use of the compounds of formula I as hereinbefore defined, in the manufacture of a medicament for treatment of 5-HT6 mediated disorders, Alzheimer's disease, cognitive disorders, cognitive impairment associated with schizophrenia, obesity and/or Parkinson's disease, and any other disorder mentioned above.

A further embodiment of the invention relates to a method of treatment of 5-HT6 mediated disorders, Alzheimer's disease, cognitive disorders, cognitive impairment associated with schizophrenia, obesity and/or Parkinson's disease, and any other disorder mentioned above, comprising administering to a mammal, including man in need of such treatment, a therapeutically effective amount of the compounds of formula I, as hereinbefore defined.

Yet another embodiment of the invention relates to a pharmaceutical composition comprising a compound of formula I as hereinbefore defined, for use in treatment of 5-HT6 mediated disorders, Alzheimer's disease, cognitive disorders, cognitive impairment associated with schizophrenia, obesity and/or Parkinson's disease, and any other disorder mentioned above.

One embodiment of the invention relates to an agent for the treatment of 5-HT6 mediated disorders, Alzheimer's disease, cognitive disorders, cognitive impairment associated with schizophrenia, obesity and/or Parkinson's disease, and any other disorder mentioned above, which comprises as active ingredient a compound of formula I as hereinbefore defined.

In the context of the present specification, the term “therapy” and “treatment” includes prevention and prophylaxis, unless there are specific indications to the contrary. The terms “treat”, “therapeutic” and “therapeutically” should be construed accordingly.

In this specification, unless stated otherwise, the terms “inhibitor” and “antagonist” mean a compound that by any means, partly or completely, blocks the transduction pathway leading to the production of a response by the agonist.

The compounds according to the present invention are modulators of the 5-HT6 receptors, and may be inhibitors, as well as agonists, inverse-agonists or partial-agonist.

The term “disorder”, unless stated otherwise, means any condition and disease associated with 5-HT6 receptor activities.

Non-Medical Use

In addition to their use in therapeutic medicine, the compounds of formula I, or salts, solvates or solvated salts thereof, are also useful as pharmacological tools in the development and standardisation of in vitro and in vivo test systems for the evaluation of the effects of modulators of 5-HT6 related activity in laboratory animals such as cats, dogs, rabbits, monkeys, rats and mice, as part of the search for new therapeutics agents.

### Examples

General Methods

The invention will now be illustrated by the following Examples in which, generally:

operations were carried out at ambient or room temperature, i.e. in the range 17 to 25° C. and under an atmosphere of an inert gas such as argon unless otherwise stated. All solvents used were analytical grade and commercially available anhydrous solvents were used for reactions;

evaporations were carried out by rotary evaporation in vacuo and work-up procedures were carried out after removal of residual solids by filtration;

HPLC analyses were performed on an Agilent HP 1000 system consisting of G1379A Micro Vacuum Degasser, G1312A Binary Pump, G1367A Wellplate auto-sampler, G1316A Thermostatted Column Compartment and G1315B Diode Array Detector. Column: X-Terra MS, Waters, 4.6×50 mm, 3.5 μm. The column temperature was set to 40° C. and the flow rate to 1.5 ml/min. The Diode Array Detector was scanned from 210-300 nm, step and peak width were set to 2 nm and 0.05 min, respectively. A linear gradient was applied, run from 0% to 100% acetonitrile, in 4 min. Mobile phase: acetonitrile/10 mM ammonium acetate in 5% acetonitrile in MilliQ Water;

thin layer chromatography (TLC) was performed on Merck TLC-plates (Silica gel 60 F₂₅₄) and UV visualized the spots. Flash chromatography was preformed on a Combi Flash® Companion™ using RediSep™ normal-phase flash columns or on Merck Silica gel 60 (0.040-0.063 mm). Typical solvents used for flash chromatography were mixtures of chloroform/methanol, toluene/ethyl acetate and ethyl acetate/hexanes;

¹H and ¹³C NMR spectra were recorded at 400 MHz for proton and 100 MHz for carbon-13 either on a Varian Unity+ 400 NMR. Spectrometer equipped with a 5mm BBO probe with Z-gradients, or a Bruker Avance 400 NMR spectrometer equipped with a 60 μl dual inverse flow probe with Z-gradients, or a Bruker DPX400 NMR spectrometer equipped with a 4-nucleus probe equipped with Z-gradients. The following reference signals were used: the middle line of DMSO-d₆ δ 2.50 (¹H); the middle line of CD₃OD δ 3.31 (¹H); acetone-d₆ 2.04 (¹H); and CDCl₃ δ 7.26 (¹H) (unless otherwise indicated);

mass spectra were recorded on a Waters LCMS consisting of an Alliance 2795 (LC), Waters PDA 2996 and a ZQ single quadrupole mass spectrometer. The mass spectrometer was equipped with an electrospray ion source (ESI) operated in a positive or negative ion mode. The capillary voltage was 3 kV and cone voltage was 30 V. The mass spectrometer s was scanned between m/z 100-700 with a scan time of 0.3 s. Separations were performed on either Waters X-Terra MS C8 (3.5 μm, 50 or 100 mm×2.1 mm i.d.) or an ACE 3 AQ (100 mm×2.1 mm i.d.) obtained from ScantecLab. Flow rates were regulated to 1.0 or 0.3 mL/min, respectively. The column temperature was set to 40° C. A linear gradient was applied using a neutral or acidic mobile phase system, starting at 100% A (A: 95:5 10 mM NH₄OAc:MeCN, or 95:5 8 mM HCOOH:MeCN) ending at 100% B (MeCN).

Alternatively, mass spectra were recorded on a Waters LCMS system (Sample Manager 2777C, 1525μ binary pump, 1500 Column Oven, ZQ, PDA2996 and ELS detector, Sedex 85). Separation was performed using a Zorbax column (C8, 3.0×50 mm, 3 μm) supplied by Agilent Tehcnologies. A four minutes linear gradient was used starting at 100% A (A: 95:5 10 mM NH₄OAc:MeOH) and ending at 100% B (MeOH). The ZQ was equipped with a combined APPI/APCI ion source and scanned in the positive mode between m/z 120-800 using a scan time of 0.3 s. The APPI repeller and the APCI corona were set to 0.86 kV and 0.80 μA, respectively. In addition, the desolvation temperature (300° C.), desolvation gas (400 L/Hr) and cone gas (5 L/Hr) were constant for both APCI and APPI mode;

preparative chromatography was run on a Gilson auto-preparative HPLC with a diode array detector. Column: XTerra MS C8, 19×300 mm, 7 μm. Gradients with MeCN and (95:5 0.1M NH₄OAc:MeCN) were used. Flow rate: 20 ml/min. Alternatively, purification was achieved on a semi preparative Shimadzu LC-8A HPLC with a Shimadzu SPD-10A UV-vis.-detector equipped with a Waters Symmetry® column (C18, 5 μm, 100 mm×19 mm). Gradients with MeCN and (95:5 0.1M NH₄OAc:MeCN) were used. Flow rate: 10 ml/min;

GC-MS analysis was performed on a CIC-MS (GC 6890, 5973N MSD) supplied by Agilent Technologies. The column used was a DB-5 MS, ID 0.25 mm×30 m, 0.25 μm. A linear temperature gradient was applied starting at 40° C. (hold 1 min) and ending at 300° C. (hold 1 min), 25° C./minute. The MS was equipped with a CI ion source and the reactant gas was methane. The MS was scanned between m/z 50-500 and the scan speed was set to 3.25 scan/s. Alternatively mass spectra (EI-DI) were recorded on a Finigan MAT SSQ 710 spectrometer;

microwave heating was performed in a Creator, Initiator or Smith Synthesizer Single-mode microwave cavity producing continuous irradiation at 2450 MHz;

yields, where present, are not necessarily the maximum attainable;

intermediates were not necessarily fully purified but their structures and purity were assessed by thin layer chromatographic, HPLC, infra-red (IR), MS and/or NMR analysis;

is the following abbreviations have been used:

HPLC high performance liquid chromatography

LC liquid chromatography

MS mass spectometry

TFA trifluoroacetic acid

DMF dimethyformamide

DIPEA N,N-diisopropylethylamine

DMSO dimethylsulfoxide

NMP 1-methyl-2-pyrrolidinone

THF tetrahydrofuran

MeOH methanol

RT room temperature

PS-DIEA Polystyrene-bound diethylamine

PG Protecting Group

PS-Trisaraine tris-(2-aminoethyl)-amine polystyrene

EtOAc ethyl acetate

Throughout the following description of such processes it is understood that, where appropriate, suitable protecting groups will be added to, and subsequently removed from, the various reactants and intermediates in a manner that will be readily understood by one skilled in the art of organic synthesis. The specific sequence of reactions depicted is not critical. For many of the compounds described the order of the reaction steps may be varied.

The invention will now be illustrated by the following non-limiting examples.

Starting materials used were either available from commercial sources or prepared according to literature procedures.

### Example 1

### (i): N-(3-Bromophenyl)-9-chloro-4-isopropyl-2,3,4,5-tetrahydro-1,4-benzoxazepine-7-sulfonamide

9-Chloro-4-isopropyl-2,3,4,5-tetrahydro-1,4-benzoxazepine-7-sulfonyl chloride (16 mg, 0.049 mmol) was dissolved in dichloromethane (0.5 ml) and acetonitrile (0.5 ml). 3-Bromoaniline (11 μl, 0.10 mmol) and pyridine (8 μl, 0.10 mmol) were added and the mixture was stirred at ambient temperature for 30 min. The solvent was evaporated and the residue was purified by preparative HPLC to give the acetate of the title compound as a solid (21 mg, 92%).

¹H NMR (400 MHz, CHLOROFORM-d) δ ppm 7.72 (1H, d) 7.48-7.52 (1H, m) 7.25-7.28 (2H, m) 7.11-7.17 (1H, m) 7.04-7.08 (1H, m) 4.18-4.23 (2H, m) 3.77 (2H, s) 3.08-3.13 (2H, m) 2.90-2.99 (1H, m) 1.08 (6H, d); MS ESI m/z M+H⁺ 459, 461; M−H⁺ 457, 459.

### (ii) 2-[(3-Chloro-2-fluorobenzyl)(isopropyl)amino]ethanol

2-(i-Propylamine)ethanol (1.83 ml, 15.8 mmol) and acetic acid (0.90 ml, 15.8 mmol) were dissolved in anhydrous THF (40 ml) and the mixture was cooled to 0° C. 3-Chloro-2-fluorobenzaldehyde (1.85 ml, 15.8 mmol) and sodium triacetoxyborohydride (5.0 g, 23.7 mmol) were added. The mixture was stirred at ambient temperature for 20 h. Saturated aqueous sodium hydrogen carbonate (8 ml) was added and the mixture was extracted with EtOAc. The organic phase was dried (MgSO₄) and the solvent was evaporated to give the title compound (3.9 g) that was used without further purification. MS ESI m/z M+H⁺ 246, 248.

### (iii) 9-Chloro-4-isopropyl-2,3,4,5-tetrahydro-1,4-benzoxazepine

2-[(3-Chloro-2-fluorobenzyl)(isopropyl)amino]ethanol (3.9 g, 15.8 mmol) was dissolved in THF:DMF (2:1, 100 ml) and the solution was added dropwise to a slurry of sodium hydride (0.80 g, 31.5 mmol) in THF:DMF (2:1, 75 ml). The reaction mixture was stirred at ambient temperature for 30 min and at 50° C. for 2.5 h. Methanol was added dropwise to quench the reaction. The mixture was neutralized with Dowex® (H⁺) resins and the resins were removed by filtration. The mixture was concentrated by evaporation. Water (50 ml) was added followed by aqueous sodium hydroxide (1M) until pH 10 was reached. The mixture was extracted with diethyl ether. The organic phase was dried (MgSO₄), evaporated and the residue was purified by preparative HPLC to give the title compound (0.64 g, 18%). MS ESI m/z M+H⁺ 226, 228.

### (iv) 9-Chloro-4-isopropyl-2,3,4,5-tetrahydro-1,4-benzoxazepine-7-sulfonyl chloride

9-Chloro-4-isopropyl-2,3,4,5-tetrahydro-1,4-benzoxazepine (100 mg, 0.443 mmol) was dissolved in chloroform (2 ml) and cooled to −15° C. Chlorosulfonic acid (0.13 ml, 1.9 mmol) in chloroform (2 ml) was added dropwise. The mixture was stirred at −15° C. for 20 min and then at RT for 20 min. The mixture was poured onto a mixture of ice, dichloromethane and sodium hydrogen carbonate (0.7 g) and was extracted with chloroform (×3). The combined organic phases were dried (MgSO₄) and the solvent was evaporated to give the title compound (16 mg, 11%) that was used directly in the next step.

### Example 2

### (i): 9-Chloro-N-(3-chlorophenyl)-2,3,11,11a-tetrahydro-1H,5H-pyrrolo[2,1-c][1,4]benzoxazepine-7-sulfonamide

9-Chloro-2,3,11,11a-tetrahydro-1H,5H-pyrrolo[2,1-c][1,4]benzoxazepine-7-sulfonyl chloride (58 mg, 0.18 mmol), 3-chloroaniline (0.10 ml, 0.98 mmol) and pyridine (79 μl, 0.98 mmol) were dissolved in chloroform:acetonitrile (2:1, 2.5 ml). The mixture was stirred for 1 h under argon atmosphere. The solvents were evaporated and the residue was purified by preparative HPLC to give the title compound (46 mg, 64%). ¹H NMR (400 MHz, CHLOROFORM-d) δ ppm 7.73 (1H, d) 7.54 (1H, d) 7.17-7.22 (1H, m) 7.08-7.14 (2H, m) 6.93-6.98 (1H, m) 4.50 (1H, dd) 3.72-3.85 (2H, m) 3.58 (1H, dd) 3.07-3.14 (1H, m) 2.85-2.94 (1H, m) 2.52-2.60 (1H, m) 1.79-2.01 (3H, m) 1.39-1.51 (1H, m); MS ESI m/z M+H⁺ 404, 406.

### (ii) [1-(3-Chloro-2-fluorobenzyl)pyrrolidin-2-yl]methanol

3-Chloro-2-fluorobenzaldehyde (1.80 ml, 15.8 mmol), D,L-prolinol (1.55 ml, 15.8 mmol) and acetic acid (0.90 ml, 15.8 mmol) were dissolved in THF (40 ml) and the mixture was cooled to 0° C. Sodium triacetoxyborohydride (5.0 g, 23.7 mmol) was added portionwise. The mixture was stirred at room temperature for 4 h. Saturated aqueous sodium hydrogen carbonate was added until the gas evolution ceased. EtOAc (90 ml) was added and the mixture was washed with water. The organic phase was dried (MgSO₄) and the solvent was evaporated to give a crude that was used directly in the next step. MS ESI m/z M+H⁺ 244, 246.

### 9-Chloro-2,3,11,11a-tetrahydro-1H,5H-pyrrolo[2,1-c][1,4]benzoxazepine

[1-(3-Chloro-2-fluorobenzyl)pyrrolidin-2-yl]methanol (crude from example 2(ii), >16 mmol) was dissolved in THF (60 ml) and was added dropwise to sodium hydride (0.76 g, 32 mmol) in THF (15 ml). The mixture was stirred at ambient temperature for 1 h. Sodium hydride (0.76 g, 31.5 mmol) was added portionwise and the mixture was heated at 50° C. for 2.5 h. The mixture was cooled to 0° C. and methanol (70 ml) was added carefully. The mixture was neutralized by addition of Dowex® (H⁺) resin. The resins were removed by filtration and the solvent was evaporated. The residue was dissolved in EtOAc and the mixture was washed with brine and water. The organic phase was dried (MgSO₄) and the solvent was evaporated. The residue was purified by preparative HPLC to give the title compound. MS ESI m/z M+H⁺ 224, 226.

### (iv): 9-Chloro-2,3,11,11a-tetrahydro-1H,5H-pyrrolo[2,1-c][1,4]benzoxazepine-7-sulfonyl chloride

9-Chloro-2,3,11,11a-tetrahydro-1H,5H-pyrrolo[2,1-c][1,4]benzoxazepine (186 mg, 0.83 mmol) was dissolved in chloroform (1.5 ml) and the mixture was cooled to −15° C. under argon atmosphere. Chlorosulfonic acid (0.24 ml, 3.57 mmol) in chloroform (1.5 ml) was to added and the mixture was stirred at −15-0° C. for 30 min and then at RT for 30 min. The mixture was poured onto ice and was extracted with chloroform. The organic phase was washed with saturated aqueous sodium hydrogen carbonate, dried (MgSO₄) and the solvent was evaporated to give a solid (117 mg, 44%) that was used directly in the next step.

### Example 3

### (i): N-(3-Chlorophenyl)-2,3,4,5-tetrahydro-1,4-benzoxazepine-7-sulfonamide

5-Oxo-2,3,4,5-tetrahydro-1,4-benzoxazepine-7-sulfonyl chloride (71 mg, 0.27 mmol) was suspended in chloroform: acetonitrile (2:1, 4 ml) and pyridine (131 μl, 1.62 mmol) and 3-chloroaniline (144 μl, 1.36 mmol) were added. The mixture was stirred at ambient temperature for 1 h and water (5 ml) was added. The mixture was extracted with chloroform, the organic layer was dried (MgSO₄) and the solvent was removed. The residue was suspended in THF (0.5 ml) and borane (1M in THF, 1.68 ml, 1.68 mmol) was added. The mixture was heated at reflux for 4 h. The mixture was cooled to 0° C. and hydrochloric acid (4 M, 0.5 ml) was added. The mixture was heated at reflux for 1 h and the mixture was concentrated in vacuo. The residue was diluted with water and sodium hydrogen carbonate was added until basic pH was reached. The mixture was extracted with chloroform (×2), the organic layer was dried (MgSO₄) and the solvent was evaporated. The residue was purified by preparative HPLC to give the title compound (5 mg, 5%). ¹H NMR (400 MHz, CHLOROFORM-d) δ ppm 7.96 (1H, br. s.) 7.62 (1H, dd) 6.96-7.16 (5H, m) 4.36 (2H, br. s.) 4.26-4.31 (2H, m) 3.52-3.58 (2H, m); MS ESI m/z M+H⁺ 339, 341.

### (ii) 3,4-Dihydro-1,4-benzoxazepin-5(2H)-one

4-Chromanone (25 g, 169 mmol) and sodium azide (33.2 g, 510 mmol) were dissolved in acetic acid (335 ml). The solution was cooled to 0° C. and concentrated sulfuric acid (50 ml) was added dropwise. The mixture was heated at reflux for 4 h and then cooled to RT. The mixture was poured onto ice (500 ml) and concentrated ammonium hydroxide was added until basic pH was reached. The mixture was stirred at ambient temperature for 20 h and the solid formed was collected by filtration to give the title compound (15 g, 54%). MS ESI m/z M+H⁺ 164.

### 5-Oxo-2,3,4,5-tetrahydro-1,4-benzoxazepine-7-sulfonyl chloride

3,4-Dihydro-1,4-benzoxazepin-5(2H)-one (150 mg, 0.92 mmol) was dissolved in chloroform (2 ml) and the mixture was cooled to −15° C. Chlorosulfonic acid (0.26 ml, 4.0 mmol) in chloroform (2 ml) was added under argon atmosphere. The mixture was stirred at −15-0° C. for 30 min and at RT for 30 min. The mixture was poured onto ice and extracted with chloroform. The organic layer was washed with aqueous sodium hydrogen carbonate, dried (MgSO₄) and the solvent was evaporated to give a solid (71 mg, 29%) that was used directly in the next step.

### Example 4

### N-(2,3-Dichlorophenyl)-2,3,4,5-tetrahydro-1,4-benzoxazepine-7-sulfonamide

The method described in example 3(i) was used to prepare the title compound (30 mg, 31%).

¹H NMR (400 MHz, CHLOROFORM-d) δ ppm 7.13-7.19 (2H, m) 7.04 (1H, dd) 6.84 (1H, dd) 6.73-6.79 (1H, m) 6.63 (1H, d) 3.64-3.69 (2H, m) 3.48 (2H, s) 2.71-2.76 (2H, m); MS ESI m/z M+H⁺ 373, 375.

### Example 5

### N-(4-Chloro-1-naphthyl)-2,3,4,5-tetrahydro-1,4-benzoxazepine-7-sulfonamide

The method described in example 3(i) was used to prepare the title compound (0.3 mg, 0.3%).

¹H NMR (400 MHz, MeOH) δ ppm 7.83 (1H, d) 7.59 (1H, d) 7.06-7.22 (5H, m) 6.82 (1H, d) 6.61 (1H, d) 3.64-3.69 (2H, m) 3.43 (2H, s) 2.73-2.79 (2H, m); MS ESI m/z M+H⁺ 389, 391.

### Example 6

### (i) 2-Bromophenyl 2,3,4,5-tetrahydro-1H-2-benzazepine-8-sulfonate

2-bromophenyl 1-oxo-2,3,4,5-tetrahydro-1H-2-benzazepine-8-sulfonate (85 mg, 0.214 mmol) was dissolved in BH₃.THF (1N, 6 ml) and the obtained reaction mixture was refluxed for 4 h. The reaction mixture was cooled to 0° C. and hydrochloric acid (4 N, 6 ml) was cautiously added. The solution was heated to reflux for 1 h. The mixture was concentrated under reduced pressure. Water (20 ml) was added and the reaction mixture was made basic by adding solid sodium hydrogencarbonate. The aqueous layer was extracted with ethyl acetate/methylenechloride (1:1) twice. The organic layer was concentrated under reduced pressure and the residue was purified by preperative HPLC to give the product (27 mg, 34%). ¹H NMR (400 MHz, CDCl₃) δ ppm 7.65-7.72 (2H, m) 7.54 (1H, dd) 7.27-738 (3H, m) 7.09-7.19 (1H, m) 4.00 (2H, s) 3.20- 3.30 (2H, m) 3.04 (2H, m) 1.73-1.82 (2H, m); MS (ESI) m/z M+H⁺ 382 and 384.

### (ii) 2-bromophenyl 1-oxo-2,3,4,5-tetrahydro-1H-2-ben 7azepine-8-sulfonate

o-Bromophenol (93 μl, 0.8 mmol) was added at room temperature to a solution of 1-oxo-2,3,4,5-tetrahydro-1H-2-benzazepine-8-sulfonyl chloride (95 mg, 0.37 mmol) and pyridine (70 μl, 0.93 mmol) in acetonitrile/methylenechloride (2:1, 2 ml). The mixture was stirred for 16 hours, diluted with methylenchloride (10 ml) and washed with water. The organic layer was dried (sodium sulfate) and concentrated at reduced pressure. The residue was purified using preperative HPLC to give the product (85 mg, 58%). MS ESI m/z M+H⁺ 396, 398.

### (iii) 1-oxo-2,3,4,5-tetrahydro-1H-2-benzazepine-8-sulfonyl chloride

2,3,4,5-tetrahydro-1H-2-benzazepin-1-one (240 mg, 1.49 mmol) was added to chlorosulfonic acid (13 ml) at 0° C. The mixture was allowed to reach room temperature and was stirred for additional 8 h. The brown solution was poured on ice and the mixture was extracted with ethyl acetate twice. The organic combined organic layer was washed with sodium hydrgencarbonate solution (5%), brine and dried over sodium sulfate. The solvent was removed under reduced pressure and the crude product (378 mg, 97%) was used in the following reaction step without further purification. MS ESI m/z M+H⁺ 260, 262.

Pharmacology

Method for [125I]SB258585 Binding to Rat Striatal 5-HT6 Receptors

Materials

[¹²⁵I]SB258585 (1) with specific radioactivity 2000 Ci/mmol was purchased from Amersham Biosciences Europe GmbH, Freiburg, Germany. Other chemicals were is purchased from commercial sources and were of analytical grade.

Preparation of Membranes

Striatal tissue from adult rats (Sprague-Dawley, 320-370 g, B & K Sweden) were dissected out, weighed and homogenized in buffer containing 50 mM Tris-HCl, 4 mM MgCl2, 1 mM EDTA, 10 μM pargyline and protease inhibitor (Complete, Roche Diagnostics) pH 7.4 using an Ultra-Turrax T8 (IKA Labortechnik, Germany). The tissue homogenate was centrifuged at 48 000×g for 10 min and the pellet was resuspended and recentrifuged as above. The final membranes were diluted in buffer to a concentration of 60 mg original wet weight (w.w.) per ml and stored in aliquots at −70° C.

Radioligand Binding Assays

Saturation binding studies were carried out in duplicate with 1-3 mg w.w. per tube in 0.5 ml buffer (50 mM Tris, 4 mM MgCl2, 100 mM NaCl, 1 mM EDTA, 5 mM ascorbate and 10 μM pargyline at pH 7.4), 0.2 nM [¹²⁵I]SB258585 and unlabelled SB258585 to give a final concentration range of 0.23-20 nM (12 conc.). Non-specific binding was determined in the presence of 10 μM methiothepin. In the competition experiments 0.8-2 mg w.w. per tube and a radioligand concentration of 0.5-1 nM were used with 7 concentrations of the competing drug pre-dissolved in DMSO and diluted in buffer. The assays were incubated for 1-3 hours at room temperature, and terminated by rapid filtration through Whatman GF/B filters pretreated with 0.3% polyethyleneimine using a Brandel cell harvester. The radioactivity was determined in a Packard Tri-Carb 2900TR liquid scintillation counter: Data were analyzed by non-linear regression analyses using PRISM 4.00 (GraphPad Software Inc., San Diego, Calif.).

More informantion about the asay can be found in Hirst, W. D., Minton, J. A. L., Bromidge, S. M., Moss, S. F., Latter, A., Riley, G., Routledge, C., Middlemiss, D. N. & Price, G. W. (2000). Characterization of [¹²⁵I]-SB-258585 binding to human recombinant and native 5-HT6 receptors in rat, pig and human brain tissue is described in Br. J. Pharmacol., 130, 1597-1605.

Results

Typical IC₅₀ values as measured in the assays described above are 5 μM or less. In one aspect of the invention the IC₅₀ is below 500 nM. In another aspect of the invention the IC₅₀ is below 50 nM. In a further aspect of the invention the IC₅₀ is below 10 nM.

TABLE 1
Specimen results from assay.

| Example no | Kᵢ (nM) | n |
|---|---|---|
| 3 | 82 ± 28 | 2 |

1-13. (canceled)

14. A compound having the formula I: 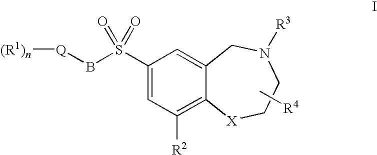
wherein:
Q is C₆₋₁₀arylC₀₋₆alkyl, C₅₋₁₁heteroarylC₀₋₆alkyl, C₃₋₇cycloalkylC₀₋₆alkyl, C₃₋₇heterocycloalkyl or C₁₋₃alkyl;
R¹ is hydrogen, hydroxyl, halogen, C₁₋₆alkyl, C₂₋₆alkenyl, C₂₋₆alkynyl, C₁₋₆alkoxy, N(R⁸)₂, C₆₋₁₀arylC₀₋₃alkyl, C₅₋₆heteroarylC₀₋₃alkyl, C₁₋₆haloalkyl, C₁₋₆haloalkylO, R⁶OC₀₋₆alkyl, CN, SR⁶, R⁶SO₂C₀₋₃alkyl, SOR⁶, R⁶CON(R⁷)C₀₋₃alkyl, NR⁷SO₂R⁶, COR⁶, COOR⁶, OSO₂R⁶, (R⁷)₂NCOC₀₋₃alkyl, SO₂N(R⁷)₂, N(R⁷)CON(R⁷)₂ or oxo;
n is 0, 1, 2, 3 or 4;
B is O, N(R⁵), or B is N in a C₅₋₁₁heteroaryl;
X is O, CH₂ or NR¹⁰;
R² is hydrogen, hydroxyl, halogen, C₁₋₆alkyl, C₁₋₆alkoxy, C₁₋₆haloalkyl, C₁₋₆haloalkylO, R⁹OC₀₋₆alkyl, CN, SR⁸, SO₂R⁹, SOR⁹, N(R⁸)COR⁹, N(R⁸)SO₂R⁹, COR⁹, COOR⁹, OSO₂R⁹, CON(R⁸)₂ or SO₂N(R⁹)₂;
R³ is hydrogen, C₁₋₁₀alkyl, C₁₋₆haloalkyl or R⁹OC₁₋₆alkyl;
R⁴ is hydrogen, C₁₋₅alkyl, C₁₋₅haloalkyl, C₁₋₅alkoxy or C₁₋₅haloalkoxy and may be substituted by one or more groups selected independently from halogen, hydroxyl, cyano, C₁₋₃alkyl and C₁₋₃alkoxy, or
R³ and R⁴ form together a C₃₋₇heterocycloalkyl, which may be substituted by one or more groups selected independently from hydrogen, halogen, C₁₋₆alkyl, C₁₋₆haloalkyl, COR⁹, SO₂R⁹, OR⁹, cyano, oxo and SO₂N(R⁸)₂;
R⁵ is hydrogen, C₁₋₆alkyl, R⁹OC₁₋₆alkyl, C₁₋₆haloalkyl or C₁₋₆cyanoalkyl;
R⁶ is C₁₋₆alkyl, C₆₋₁₀arylC₀₋₃alkyl, C₅₋₆heteroarylC₀₋₃alkyl, C₃₋₇cycloalkylC₀₋₃alkyl or C₁₋₃haloalkyl;
R⁷ is hydrogen, C₁₋₆alkyl, C₁₋₆haloalkyl, C₃₋₇cycloalkylC₀₋₃alkyl, C₆₋₁₀arylC₀₋₃alkyl or C₅₋₆heteroarylC₀₋₃alkyl, or
R⁶ and R⁷ form together a C₅₋₆heteroaryl or C₃₋₇heterocycloalkyl,
whereby any aryl and heteroaryl under R¹, R⁶ and R⁷ may be substituted by one or more groups selected independently from hydrogen, halogen, hydroxyl, C₁₋₆haloalkyl, CN, OR⁸, C₁₋₆alkyl, oxo, SR⁸, CON(R⁸)₂, N(R⁸)COR⁹, SO₂R⁹, SOR⁹, N(R⁸)₂ and COR⁹;
R⁸ is hydrogen, C₁₋₆alkyl, C₁₋₆cyanoalkyl or C₁₋₆haloalkyl; and
R⁹ is C₁₋₆alkyl, C₁₋₆cyanoalkyl or C₁₋₆haloalkyl;
R⁸and R⁹ form together a C₃₋₇heterocycloalkyl which may be substituted by one or more groups selected independently from hydrogen, halogen, hydroxyl, C₁₋₃alkyl, C₁₋₃alkoxy and cyano; and
R¹⁰ is H, C₁₋₆alkyl, COR¹¹ or SO₂R¹¹;
or salts, solvates or solvated salts thereof.

15. A compound according to claim 14, wherein:
Q is C₆₋₁₀arylC₀₋₆alkyl or C₅₋₁₁heteroarylC₀₋₆alkyl;
R¹ is hydrogen, halogen, C₁₋₆alkyl, C₆₋₁₀arylC₀₋₃alkyl, C₅₋₆heteroarylC₀₋₃alkyl, C₁₋₆haloalkyl, CN or R⁶OC₀₋₆alkyl;
n is 1 or 2;
B is O or N(R⁵);
X is O, CH₂ or NR¹⁰;
R² is hydrogen, hydroxyl, halogen, C₁₋₆alkyl, C₁₋₆alkoxy, C₁₋₆haloalkyl, C₁₋₆haloalkylO;
R³ is hydrogen, C₁₋₁₀alkyl, C₁₋₆haloalkyl or R⁹OC₁₋₆alkyl;
R⁴ is hydrogen, C₁₋₅alkyl, C₁₋₅haloalkyl, C₁₋₅alkoxy or C₁₋₅haloalkoxy and may be substituted by one or more groups selected independently from halogen, hydroxyl, cyano, C₁₋₃alkyl and C₁₋₃alkoxy, or
R³ and R⁴ form together a C₃₋₇heterocycloalkyl, which may be substituted by one or more groups selected independently from hydrogen, halogen, C₁₋₆alkyl, C₁₋₆COR⁹, SO₂R⁹, OR⁹, cyano, oxo and SO₂N(R⁸)₂; and
R⁵ is hydrogen, C₁₋₆alkyl, C₁₋₆haloalkyl or C₁₋₆cyanoalkyl;
or salts, solvates or solvated salts thereof.

16. A compound according to claim 14 wherein:
Q is C₆₋₁₀arylC₀₋₆alkyl;
R¹ is halogen;
n is 1 or 2;
B is O or N(R⁵);
X is O, CH₂ or N¹⁰;
R² is hydrogen or halogen;
R³ is hydrogen or C₁₋₁₀alkyl;
R⁴ is hydrogen or C₁₋₅alkyl, or
R³ and R⁴ form together a C₃₋₇heterocycloalkyl; and
R⁵ is hydrogen;
or salts, solvates or solvated salts thereof.

17. Compounds according to claim 14, said compounds selected from the group consisting of:
N-(3-Bromophenyl)-9-chloro-4-isopropyl-2,3,4,5-tetrahydro-1,4-benzoxazepine-7-sulfonamide;
9-Chloro-N-(3-chlorophenyl)-2,3,11,11a-tetrahydro-1H,5H-pyrrolo[2,1-c][1,4]benzoxazepine-7-sulfonamide;
N-(3-Chlorophenyl)-2,3,4,5-tetrahydro-1,4-benzoxazepine-7-sulfonamide;
N-(2,3-Dichlorophenyl)-2,3,4,5-tetrahydro-1,4-benzoxazepine-7-sulfonamide;
N-(4-Chloro-1-naphthyl)-2,3,4,5-tetrahydro-1,4-benzoxazepine-7-sulfonamide; and
2-Bromophenyl 2,3,4,5-tetrahydro-1H-2-benzazepine-8-sulfonate,
or salts, solvates or solvated salts thereof.

18. A pharmaceutical composition comprising as an active ingredient a therapeutically effective amount of a compound according to claim 14, in association with one or more pharmaceutically acceptable diluents, excipients and/or inert carriers.

19. A pharmaceutical composition comprising a compound according to claim 14, for use in the treatment of 5-HT6 mediated disorders.

20. A method for treating 5-HT6 mediated disorders, comprising administering to a mammal, including a human in need of such treatment, a therapeutically effective amount of a compound of formula I, according to claim 14.

21. A method of treating Alzheimer's disease, comprising administering to a mammal, including a human in need of such treatment, a therapeutically effective amount of a compound of formula I, according to claim 14.

22. A compound having the formula M1 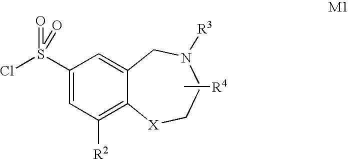
wherein X, R², R³ and R⁴ are defined as in claim 14.

23. The use of a compound according to claim 22 in the preparation of compounds of formula I according to claim 14.

24. A process for the preparation of a compound according to claim 14 or a pharmaceutically acceptable salt thereof, which process comprises using a compound of formula M1 as defined herein.
